(19) United States
(12) Patent Application Publication
(10) Pub. No.: US 2006/0160247 A1
(43) Pub. Date: Jul. 20, 2006

(54) Unsaturated carboxylic acid hemicacetal ester, polymeric compound and photoresist resin composition

(76) Inventors: Hiroshi Koyama, Himeji-shi, Hyogo (JP); Keizo Inoue, Hyogo (JP); Takahiro Iwahama, Hyogo (JP); Mari Sumida, Hyogo (JP)

Correspondence Address: BIRCH STEWART KOLASCH & BIRCH, PO BOX 747, FALLS CHURCH, VA, 22040-0747, US

(21) Appl. No.: 10/537,120

(22) PCT Filed: Jan. 17, 2005
(86) PCT No.: PCT/JP05/00794

§ 371 Date: Jun. 3, 2005

(51) Int. Cl. C08F 118/02; C07C 69/52; H01L 21/00

(52) U.S. Cl. 438/1; 526/319; 560/224

(57) ABSTRACT

A polymeric compound having a repeated unit corresponding to an unsaturated carboxylic acid hemiacetal ester represented by the following formula (1); 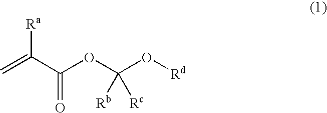
wherein R^{a} is a hydrogen atom, a halogen atom, an alkyl group of carbon number 1 to 6 or a haloalkyl group of carbon number 1 to 6, R^{b} is a hydrocarbon group having a hydrogen atom at a first poison, R^{c} is a hydrogen atom or a hydrocarbon group and R^{d} is an organic group having a cyclic skeleton. This polymeric compound, further, may have a repeated unit corresponding to at least one monomer selected from a monomer having a lactone skeleton, a monomer having a cyclic ketone skeleton, a monomer having an acid anhydride group and a monomer having an imide group [except for a repeated unit corresponding to the said unsaturated carboxylic acid hemiacetal ester] and/or a repeated unit corresponding to at least one monomer selected from a monomer having a hydroxyl group and others. This polymeric compound shows superior acid-eliminating function in case of using as photoresist.

### TECHNICAL FIELD

The present invention relates to an useful unsaturated carboxylic acid hemiacetal ester as a monomer component of a photoresist resin used for a micro processing of semiconductor and others, a polymeric compound having a repeated unit corresponding to the unsaturated carboxylic acid hemiacetal ester, a photoresist resin composition containing the polymeric compound and a process of producing a semi-conductor.

### BACKGROUND ART

A positive-type photoresist used in a process of producing a semi-conductor must have properties such as a property allowing an exposed part by light exposure to change to alkali soluble, adhesion to silicone wafer, plasma-etching resistance and transparency against used light. The positive photoresist is used as a solution containing a polymer which is a main component, a photo-acid generator and some sorts of additives for adjusting the above properties and to prepare a resist corresponding to an use, it is extremely important that a polymer as a main component has the above properties in balance.

An exposure light source of lithography using to a production of semi-conductor has become shorter wavelength year after year and KrF excimer laser of wavelength 248 nm is converting to ArF excimer laser of wavelength 193 nm. In a resist polymer used for a exposure machine of such a KrF or ArF excimer laser, an unit having a 2-methyladamantane-2-yl group or a 1-adamantyl-1-methyethyl group and others are known as a monomer unit which gives a function becoming to be soluble against an alkali developer due to elimination by an acid generated from a photo-acid generator by exposure (Japanese Unexamined Patent Application Publication No. 1997-73173). However, a conventional resist resin having these units isn't sufficient in sensitivity or developing quality. Further, a balance of substrate adhesion, etching resistance and acid-eliminating function isn't sufficient.

### DISCLOSURE OF INVENTION

An object of the present invention is to provide a polymeric compound which shows superior acid-eliminating function or superior acid-eliminating function and substrate adhesion in case of using as photoresist, a monomer thereof, a process of producing the monomer, a photoresist resin composition containing the said polymeric compound and a process of producing a semi-conductor using the resin composition.

Another object of the present invention is to provide a photoresist polymeric compound having substrate adhesion, etching resistance and acid-eliminating function in balance, a photoresist resin composition containing the polymeric compound and a process of producing a semi-conductor using the resin composition.

Further, another object of the present invention is to provide a photoresist polymeric compound which can form a micro pattern accurately, a photoresist resin composition and a process of producing a semi-conductor.

The present inventors made intensive investigations to achieve the above objects and found that superior acid-eliminating function or superior acid-eliminating function and substrate adhesion are performed and a micro pattern can be formed accurately by using a polymeric compound containing a repeated unit corresponding to an unsaturated carboxylic acid hemiacetal ester having a specific construction as a photoresist resin and the present invention was achieved.

Namely, the present invention provides an unsaturated carboxylic acid hemiacetal ester represented by the following formula (1); 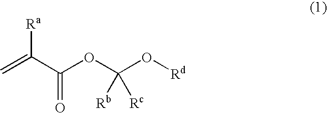
wherein R^{a} is a hydrogen atom, a halogen atom, an alkyl group of carbon number 1 to 6 or a haloalkyl group of carbon number 1 to 6, R^{b} is a hydrocarbon group having a hydrogen atom at a first poison, R^{c} is a hydrogen atom or a hydrocarbon group and R^{d} is an organic group having a cyclic skeleton.

As the said cyclic skeleton in R^{d}, a lactone skeleton or a non-aromatic polycyclic skeleton is preferable.

The present invention, further, provides a process of producing an unsaturated carboxylic acid hemiacetal ester, wherein the unsaturated carboxylic acid hemiacetal ester represented by the following formula (5); 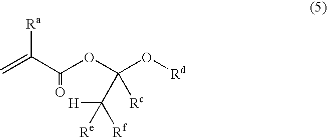
wherein R^{a} is a hydrogen atom, a halogen atom, an alkyl group of carbon number 1 to 6 or a haloalkyl group of carbon number 1 to 6, R^{c} is a hydrogen atom or a hydrocarbon group, R^{d} is an organic group having a cyclic skeleton and each of R^{e} and R^{f} is a hydrogen atom or a hydrocarbon group; is obtained by allowing an unsaturated carboxylic acid represented by the following formula (3); 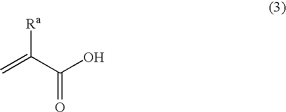
wherein R^{a} is a hydrogen atom, a halogen atom, an alkyl group of carbon number 1 to 6 or a haloalkyl group of carbon number 1 to 6;
to react with a vinyl ether compound represented by the following formula (4); 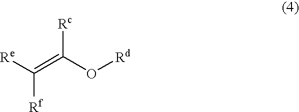
wherein R^{c} is a hydrogen atom or a hydrocarbon group, R^{d} is an organic group having a cyclic skeleton and each of R^{e} and R^{f} is a hydrogen atom or a hydrocarbon group.

The present invention, in addition, provides a polymeric compound having a repeated unit represented by the formula (I); 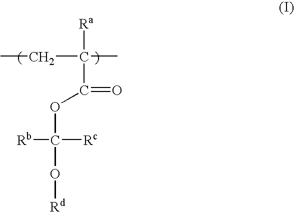
wherein R^{a} is a hydrogen atom, a halogen atom, an alkyl group of carbon number 1 to 6 or a haloalkyl group of carbon number 1 to 6, R^{b} is a hydrocarbon group having a hydrogen atom at a first poison, R^{c} is a hydrogen atom or a hydrocarbon group and R^{d} is an organic group having a cyclic skeleton.

In addition, this polymeric compound may have a repeated unit corresponding to at least one monomer selected from a monomer having a lactone skeleton, a monomer having a cyclic ketone skeleton, a monomer having an acid anhydride group and a monomer having an imide group; provided that except for a repeated unit represented by the formula (I). In addition, the said polymeric compound may have a repeated unit corresponding to at least one monomer selected from a monomer having a hydroxyl group, a monomer having a mercapto group and a monomer having a carboxyl group.

Further, the present invention provides a photoresist resin composition containing at least the said polymeric compound and a photo-acid generator.

The present invention, further, provides a process of producing a semi-conductor comprising steps of coating the said photoresist resin composition on a base or substrate to form a resist film and forming a pattern through exposure and development.

Further, in the present description a vinyl ether monomer and a vinyl ether compound also include a compound in which a hydrogen atom of vinyl group is substituted by a substituent. In addition, as a protecting group of a hydroxyl group an others, a common protecting group in an organic synthesis field can be used.

Due to the present invention, a polymeric compound which shows superior acid-eliminating function or superior acid-eliminating function and substrate adhesion and a monomer thereof when used as a photoresist are provided. In addition, a photoresist resin composition of the present invention is superior for acid-eliminating and further performs substrate adhesion, etching resistance and acid-eliminating function in balance. Thereby a micro pattern can be formed accurately in a semi-conductor production.

### BEST MODE FOR CARRYING OUT THE INVENTION

[Unsaturated Carboxylic Acid Hemiacetal Ester]

An unsaturated carboxylic acid hemiacetal ester of the present invention is presented by the above formula (1). In the formula (1), R^{a} is a hydrogen atom, a halogen atom, an alkyl group of carbon number 1 to 6 or a haloalkyl group of carbon number 1 to 6, and R^{b} is a hydrocarbon group having a hydrogen atom at 1st position, R^{c} is a hydrogen atom or a hydrocarbon group and R^{d} is an organic group having a cyclic skeleton.

In a halogen atom of the above R^{a}, a fluorine atom, a chlorine atom, a bromine atom and others are included. As an alkyl group of carbon number 1 to 6, there may be mentioned, for example, a methyl, an ethyl, a propyl, an isopropyl, a s-butyl, a t-butyl, a pentyl, a hexyl and others. In these, an alkyl group of C₁ to C₃, particularly a methyl group, is preferable. As a haloalkyl group of carbon number 1 to 6, there may be mentioned, for example, a chloroalkyl group such as a chloromethyl group; a fluoroalkyl group such as a trifluoromethyl, a 2,2,2-trifluoroethyl and a pentafluoroethyl group (preferably a fluoroalkyl group of C₁ to C₃); and others.

As a hydrocarbon group having a hydrogen atom at 1st position of the said R^{b}, there may be mentioned, for example, an alkyl group such as a methyl, an ethyl, a propyl, an isopropyl, a butyl, an isobutyl and a s-butyl group (for example, an alkyl group of C₁ to C₆, particularly an alkyl group of C₁ to C₃); a cycloalkyl group such as a cyclopropyl, a cyclopentyl and a cyclohexyl group (for example, a cycloalkyl group of 3 to 6 members); a cycloalkylalkyl group such as a cyclopentyl methyl and a cyclohexyl methyl group [for example, a mono or di-(cycloalkyl of 3 to 6 members)-C₁₋₃ alkyl group]; an aralkyl group such as a benzyl, a 1-methylbenzyl and a 1-phenylbenzyl group (for example, mono or diphenyl-C₁₋₃ alkyl group); and so on. As Rb, an alkyl group of C₁ to C₃ such as a methyl, an ethyl, a propyl and an isopropyl group is preferable, particularly a methyl group is preferable.

As a hydrocarbon group of R^{c}, there may be mentioned, for example, an alkyl group such as a methyl, an ethyl, a propyl, an isopropyl, a butyl, an isobutyl and a s-butyl group (for example, an alkyl group of C₁ to C₆, particularly an alkyl group of C₁ to C₃); a cycloalkyl group such as a cyclopropyl, a cyclopentyl and a cyclohexyl group (for example, a cycloalkyl group of 3 to 6 members); an aryl group such as a phenyl group; and so on. As R^{c}, an alkyl group of C₁ to C₃ such as a methyl, an ethyl, a propyl and an isopropyl group is preferable and particularly a hydrogen atom and a methyl group are preferable.

As a cyclic skeleton of an organic group having the cyclic skeleton, there may be mentioned, a lactone skeleton and a cyclic skeleton except for the lactone skeleton.

In a lactone skelton, a skeleton constituted by only a lactone ring (for example, γ-butyrolactone ring, δ-valerolactone ring, ε-caprolactone ring and others) and further a skeleton in which the lactone ring is condensed by a non-aromatic or aromatic carbon ring or hetero ring are included. Especially, a ring constituted by only a lactone ring and a skeleton in which the lactone ring is condensed by a non-aromatic carbon ring or hetero ring (particularly a non-aromatic carbon ring) are preferable. A ring constituting a lactone skeleton may have a substituent such as an alkyl group such as a methyl group (for example, an alkyl group of C₁ to C₄ and others), a haloalkyl group such as a trifluoromethyl group (for example, a haloalkyl group of C₁ to C₄ and others), a halogen atom such as a chlorine atom and a fluorine atom, a hydroxyl group which may be protected by a protecting group, a hydroxylalkyl group which may be protected by a protecting group, a mercapto group which may be protected by a protecting group, a carboxyl group which may be protected by a protecting group, an amino group which may be protected by a protecting group, a sulfonic acid group which may be protected by a protecting group. As a protecting group, there may be mentioned a protecting group usually used in an organic synthesis field.

As a typical lactone skeleton, there may be mentioned a skeleton (group) represented by the following formulae (6a), (6b), (6c), (6d), (6e), (6f) and (6g) 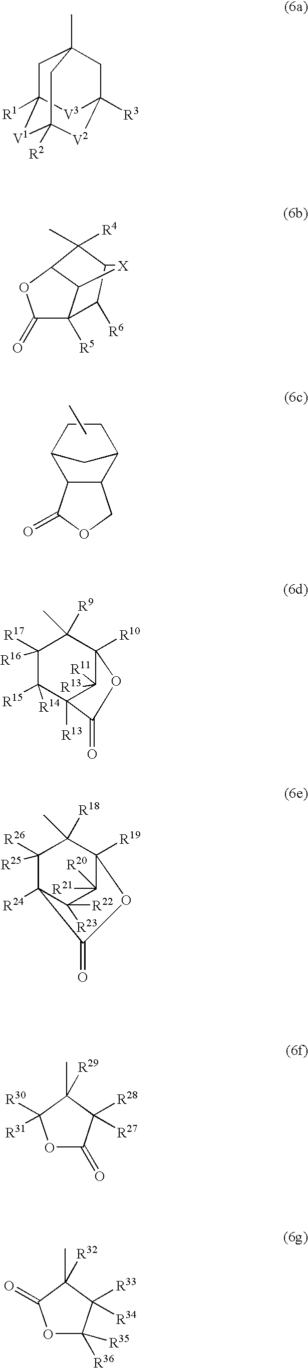
[wherein each of R¹ to R⁶ and R⁹ to R³⁶ is identical to or different from a hydrogen atom, a halogen atom, an alkyl group, a haloalkyl group, a hydroxyl group which may be protected by a protecting group, a hydroxylalkyl group which may be protected by a protecting group, a mercapto group which may be protected by a protecting group or a carboxyl group which may be protected by a protecting group, X is an alkylene group, an oxygen atom, a sulfur atom or non-bonding, and each of V¹ to V³ is identical to or different from —CH₂—, —CO— or —COO—. Provided that at least one of V¹ to V³ is —COO—. In the formula (6f), at least two groups of R²⁷ to R³¹ may be bonded together to form a ring with a carbon atom or a carbon-carbon bond. Further, in the formula (6g), at least two groups of R³² to R³⁶ may be bonded together to form a ring with a carbon atom or a carbon-carbon bond.]

In the formulae (6a) to (6g), as a halogen atom of R¹ to R⁶ and R⁹ to R³⁶, there may be mentioned a fluorine atom, a chlorine atom and soon. As an alkyl group, there may be mentioned a linear or branched chain alkyl group of carbon number 1 to 13 such as a methyl, an ethyl, a propyl, an isopropyl, a butyl, an isobutyl, a s-butyl, a t-butyl, a hexyl, an octyl, a decyl and a dodecyl group, and so on. In these, an alkyl group of carbon number 1 to 4 is preferable. As a haloalkyl group, there may be mentioned a fluoroalkyl group of carbon number 1 to 13 such as a trifluoromethyl and a pentafluoroethyl group, and so on. As a hydroxyl group which may be protected by a protecting group, there may be mentioned, for example, a hydroxyl group, a substituted oxy group (for example, an alkoxy group of C₁ to C₄ such as a methoxy, an ethoxy and a propoxy group, and others) and so on. As a hydroxyalkyl group which may be protected by a protecting group, there may be mentioned a group in which the said hydroxyl group which may be protected by a protecting group is bonded through an alkylene group of carbon number 1 to 6 and soon. As a mercapto group which may be protected by a protecting group, there may be mentioned a mercapto group and further a mercapto group protected by the same protecting group as the said hydroxyl group, and so on. As a carboxyl group which may be protected by a protecting group, there may be mentioned a —COOR^{y} group and so on. The said R^{y} is a hydrogen atom or an alkyl group and as the alkyl group, there may be mentioned a linear or branched chain alkyl group of carbon number 1 to 6 such as a methyl, an ethyl, a propyl, an isopropyl, a butyl, an isobutyl, a s-butyl, a t-butyl, a hexyl group, and so on. As an alkylene group of X, there may be mentioned a linear or branched chain alkylene group of carbon number about 1 to 3 (preferably 1 or 2) such as a methylene, a dimethylmethylene, an ethylene, a propylene, a trimethylene group, and so on. As a ring in which at least two group of R²⁷ to R³¹ are bonded together to form with a carbon atom or a carbon-carbon bond and a ring in which at least two group of R³² to R³⁶ are bonded together to form with a carbon atom or a carbon-carbon bond, there may be mentioned an alicyclic carbon ring (a bridged carbon ring is included) such as a cyclopentane ring, a cyclohexane ring and a norbornane ring, and so on. A ring constituting a skeleton represented by the formulae (6a) to (6g) may have a substituent as above.

In a “ring” constituting a cyclic skeleton except for the said lactone skeleton, a non-aromatic or aromatic ring of a single or multiple ring is included. As a non-aromatic ring of a single ring, there may be mentioned, for example, an alicyclic ring such as a cycloalkane ring of about 3 to 15 members such as a cyclopentane ring, a cyclohexane ring, a cyclooctane ring and a cyclodecane ring; a non-aromatic hetero ring of about 3 to 15 members such as a tetrahydrofuran ring, a pyrrolidine ring, piperidine ring and a morpholine ring, and so on. As a non-aromatic polycyclic ring, there may be mentioned, for example, an adamantane ring; a ring having a norbornane ring or a norbornene ring such as a norbornane ring, a norbornene ring, a bornane ring, an isobornane ring, a tricyclo[5.2.1.0^{2,6}]decane ring, a tetracyclo[4.4.0.1^{2,5}.1^{7,10}]dodecane; a ring formed by hydrogenating a polycyclic aromatic condensed ring (preferably, a ring hydrogenated perfectly) such as a perhydroindene ring, a decalin ring (a perhydronaphthalene ring), a perhydrofluorene ring (a tricyclo[7.4.0.0^{3,8}]tridecane ring) and a perhydroanthracene ring; a bridged ring of 2 cyclic, 3 cyclic and 4 cyclic (for example, a bridged carbon ring of carbon number about 6 to 20) such as a tricyclo[4.2.2.1^{2,6}]undecane; and so on. As an aromatic ring of a single ring or multiple ring, there may be mentioned an aromatic carbon ring and an aromatic hetero ring such as a benzene ring, a naphthalene ring, a pyridine ring and a quinoline ring. In these, a non-aromatic ring is preferable, more preferably a polycyclic non-aromatic ring and particularly a polycyclic non-aromatic carbon ring (a bridged ring) in view of light transparency and etching resistance and so on when used as a photoresist resin after polymerizing. In addition, in a bridged carbon ring, a ring having the said norbornane ring or a ring having the norbornane ring and a ring formed by hydrogenating an polycyclic aromatic condensed ring (particularly, a ring hydrogenated completely) are particularly preferable. Therefore, as a cyclic skeleton except for a lactone skeleton, a non-aromatic cyclic skeleton is preferable, particularly a non-aromatic polycyclic skeleton, more particularly a non-aromatic polycyclic carbon ring skeleton.

A ring constituting a cyclic skeleton except for the said lactone skeleton may have a substituent such as an alkyl group (for example, an alkyl group of C₁ to C₄ and others) such as a methyl group, a haloalkyl group (for example, a haloalkyl group of C₁ to C₄ and others) such as a trifluoromethyl group, a halogen atom such as a chlorine atom, a fluorine atom and others, a hydroxyl group which may be protected by a protecting group, a hydroxyl alkyl group which may be protected by a protecting group, a mercapto group which may be protected by a protecting group or a carboxyl group which may be protected by a protecting group, an amino group which may be protected by a protecting group, a sulfonic acid group which may be protected by a protecting group. As the protecting group, there may be mentioned a protecting group usually used in an organic synthesis field.

In an organic group containing a cyclic skeleton of R^{d}, a group represented by the following formula (2) is included. 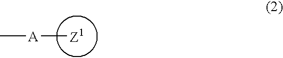
wherein A is a combining group and Z¹ is a ring constituting a cyclic skeleton.

As the said combining group in A, there may be mentioned, for example, a single bond; a linear or branched chain alkylene group such as a methylene, a methylmethylene, a dimethylmethylene, an ethylene, a propylene, a trimethylene group; a carbonyl group; an oxygen atom (an ether bond; —O—); an oxycarbonyl group (an ester bond; —COO—); an amino carbonyl group (an amide bond; —CONH—); a group constituted by the plural number of these; and so on. In preferable combining group, a single bond, a linear or branched chain alkylene group of C₁ to C₆ (particularly an alkylene group of C₁ to C₃) and others are included. The combining group may have a substituent such as, for example, a halogen atom such as a chlorine atom, a fluorine atom and others, a hydroxyl group which may be protected by a protecting group, a hydroxyl alkyl group which may be protected by a protecting group, a mercapto group which may be protected by a protecting group, a carboxyl group which may be protected by a protecting group, an amino group which may be protected by a protecting group, a sulfonic acid group which may be protected by a protecting group. As a lactone skeleton in the said cyclic skeleton of Z¹, there may be mentioned a skeleton represented by the above formulae (6a) to (6g). Further, a ring constituting a cyclic skeleton except a lactone skeleton in the cyclic skeleton of Z¹, there may be mentioned ones exemplified above.

As a typical example of a compound having an organic group containing a lactone skeleton in an unsaturated carboxylic acid hemiacetal ester represented by the formula (1) [a compound having a lactone skeleton in a compound containing a group represented by the formula (2)], there may be mentioned following compounds and however these isn't limited.
[1-1] 1-[1-(meth)acryloyloxyethoxy]-4-oxatricylo[4.3.1.1^{3,8}]uondecane-5-on [Z¹=formula (6a), A=single bond]
[1-2] 2-[1-(meth)acryloyloxyethoxy]-4-oxatricylo[4.2.1.0^{3,7}]nonane-5-on [Z¹=formula (6b), A=single bond]
[1-3] 2-[1-(meth)acryloyloxyethoxy]-6-methyl-4-oxatricyclo[4.2.1.0^{3,7}]nonane-5-on [Z¹=formula (6b), A=single bond]
[1-4] 2-[1-(meth)acryloyloxyethoxy]-6-trifluoromethyl-4-oxatricyclo[4.2.1.0^{3,7}]nonane-5-on [Z¹=formula (6b), A=single bond]
[1-5] 2-[1-(meth)acryloyloxyethoxy]-9-methyl-4-oxatricyclo[4.2.1.0^{3,7}]nonane-5-on [Z¹=formula (6b), A=single bond]
[1-6] 6-fluoro-2-[1-(meth)acryloyloxyethoxy]-4-oxatricyclo[4.2.1.0^{3,7}]nonane-5-on [Z¹=formula (6b), A=single bond]
[1-7] 9-carboxy-2-[1-(meth)acryloyloxyethoxy]-4-oxatricyclo[4.2.1.0^{3,7}]nonane-5-on [Z¹=formula (6b), A=single bond]
[1-8] 2-[1-(meth)acryloyloxyethoxy]-9-methoxycarbonyl-4-oxatricyclo[4.2.1.0^{3,7}]nonane-5-on [Z¹=formula (6b), A=single bond]
[1-9] 9-ethoxycarboxy-2-[1-(meth)acryloyloxyethoxy]-4-oxatricyclo[4.2.1.0^{3,7}]nonane-5-on [Z¹=formula (6b), A=single bond]
[1-10] 9-t-butoxycarboxy-2-[1-(meth)acryloyloxyethoxy]-4-oxatricyclo[4.2.1.0^{3,7}]nonane-5-on [Z¹=formula (6b), A=single bond]
[1-11] 2-[1-(meth)acryloyloxyethoxy]-4,8-dioxatricyclo[4.2.1.0^{3,7}]nonane-5-on [Z¹=formula (6b), A=single bond]
[1-12] 4-[1-(meth)acryloyloxyethoxy]-6-oxabicyclo[3.2.1]octane-7-on [Z¹=formula (6b), A=single bond]
[1-13] 8-[1-(meth)acryloyloxyethoxy]-4-oxatricyclo[5.2.1.0^{2,6}]decane-5-on [Z¹=formula (6c), A=single bond]
[1-14] 9-[1-(meth)acryloyloxyethoxy]-4-oxatricyclo[5.2.1.0^{2,6}]decane-5-on [Z¹=formula (6c), A=single bond]
[1-15] α-[1-(meth)acryloyloxyethoxy]-γ,γ-dimethyl-γ-butyrolactone [Z¹=formula (6g), A=single bond]
[1-16] 3-[1-(meth)acryloyloxyethoxy]-2-oxo-1-oxaspiro[4.5]decane [Z¹=formula (6g), A=single bond]
[1-17] α-[1-(meth)acryloyloxyethoxy]-γ-butyrolactone [Z¹=formula (6g), A=single bond]
[1-18] α-[1-(meth)acryloyloxyethoxy]-α,γ,γ-trimethyl-γ-butyrolactone [Z¹=formula (6g), A=single bond]
[1-19] α-[1-(meth)acryloyloxyethoxy]-β,β-dimethyl-γ-butyrolactone [Z¹=formula (6g), A=single bond]
[1-20] a compound represented by the following formula (7) [Z¹=formula (6g), A=single bond] 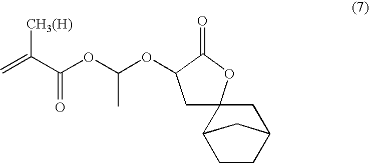
   [1-21] 3-[1-(meth)acryloyloxyethoxy]-2-oxo-1-oxaspiro[4.4]nonane [Z¹=formula (6g), A=single bond]

In an unsaturated carboxylic acid hemiacetal ester represented by the formula (1), as a typical example of compound having an organic group containing a cyclic skeleton except for a lactone skeleton [in a compound having a group represented by the formula (2), a compound having an organic group containing a cyclic skeleton except for a lactone skeleton], there may be mentioned the following compounds and however those aren't limited.
[1-22] 1-(adamantane-1-yloxy)ethyl(meth)acrylate
[1-23] 1-(adamantane-1-ylmethoxy)ethyl(meth)acrylate
[1-24] 1-[2-(adamantane-1-yl)ethoxy]ethyl(meth)acrylate
[1-25] 1-[1-(adamantane-1-yl)-1-methyethoxy]ethyl(meth)acrylate
[1-26] 1-(2-methyladamantane-2-yloxy)ethyl(meth)acrylate
[1-27] 1-(3-hydroxyadamantane-1-yloxy)ethyl(meth)acrylate
[1-28] 1-(3,5-dihydroxyadamantane-1-yloxy)ethyl(meth)acrylate
[1-29] 1-(3-carboxyadamantane-1-yloxy)ethyl(meth)acrylate
[1-30] 1-(3,5-dicarboxyadamantane-1-yloxy)ethyl(meth)acrylate
[1-31] 1-(norbornane-2-yloxy)ethyl(meth)acrylate
[1-32] 1-(norbornane-2-ylmethoxy)ethyl(meth)acrylate
[1-33] 1-(2-methylnorbornane-2-yloxy)ethyl(meth)acrylate
[1-34] 1-[1-(norbornane-2-yl)-1-methylethoxy]ethyl(meth)acrylate
[1-35] 1-(3-hydroxynorbornane-2-yloxy)ethyl(meth)acrylate
[1-36] 1-(3-hydroxymethylnorbornane-2-ylmethoxy)ethyl(meth)acrylate
[1-37] 1-(5,6-dihydroxynorbornane-2-ylmethoxy)ethyl(meth)acrylate
[1-38] 1-(3-methylnorbornane-2-ylmethoxy)ethyl(meth)acrylate
[1-39] 1-(decaline-1-yloxy)ethyl(meth)acrylate
[1-40] 1-(decaline-2-yloxy)ethyl(meth)acrylate
[1-41] 1-(5-hydroxydecaline-1-yloxy)ethyl(meth)acrylate
[1-42] 8-hydroxymethyl-4-[1-(meth)acryloyloxyethoxymethyl]tricyclo[5.2.1.0^{2,6}]decane
[1-43] 4-hydroxymethyl-8-[1-(meth)acryloyloxyethoxymethyl]tricyclo[5.2.1.0^{2,6}]decane
[1-44] 1-(bornyloxy)ethyl(meth)acrylate
[1-45] 1-(isobornyloxy)ethyl(meth)acrylate
[1-46] 3-carboxy-8-[1-(meth)acryloyloxyethoxy]tetracyclo[4.4.0.1^{2,5}.1^{7,10}]dodecane
[1-47] 3-carboxy-9-[1-(meth)acryloyloxyethoxy]tetracyclo[4.4.0.1^{2,5}1^{7,10}]dodecane

An unsaturated carboxylic acid hemiacetal ester represented by the formula (1) can be produced, for example as shown the following reaction formula, by reacting an unsaturated carboxylic acid represented by the formula (3) with a vinylether compound represented by the formula (4) in a solvent or without any solvent. A compound represented by the formula (5), which is a product, is corresponding to a compound represented by the above formula (1). 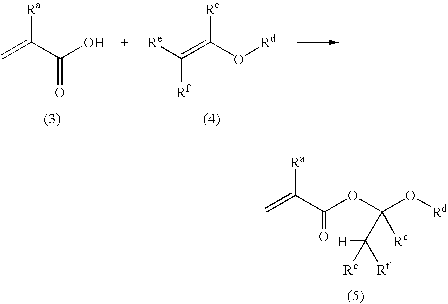
(Wherein each of R^{a}, R^{c} and R^{d} is the same as above. Each of R^{e} and R^{f} is a hydrogen atom or a hydrocarbon group, and —CHR^{e}R^{f} is corresponding to the above R^{b}.)

The above reaction is performed without any catalyst and further the reaction can be accelerated by using a catalyst. An acid catalyst isn't limited particularly and either an inorganic acid or an organic acid can be applied. As an inorganic acid, there may be mentioned, for example, a mineral acid such as hydrochloric acid, sulfuric acid, nitric acid, phosphoric acid and boric acid; a hetero poly acid such as phosphomolybdic acid, silicomolybdic acid, phosphotungstic acid and silicotungstic acid; a solid catalyst such as zeolite; and others. As an organic acid, there may be mentioned, for example, a carboxylic acid such as formic acid, acetic acid, trifluoro acetic acid; a sulfonic acid such as methane sulfonic acid, trifluoromethane sulfonic acid, benzen sulfonic acid, p-toluene sulfonic acid and naphthalene sulfonic acid; and others. As an acid catalyst, a cation exchange resin may be used. Further, a Lewis acid can be used. In addition, a substance which can be allowed to form a salt in the above acids can be used as a pyridinium salt, an ammonium salt, an alkali metal salt, an alkali-earth metal salt, a transition metal salt thereof and others. In these, phosphoric acid is particularly preferable due to a yield and selectivity of target compound.

As a solvent, it isn't especially limited if it is an inactive solvent toward the reactions and there may be mentioned, for example, an aliphatic hydrocarbon such as hexane and octane; an aromatic hydrocarbon such as benzene, toluene and xylene; an alicyclic hydrocarbon such as cyclohexane and methylcyclohexane; a halogenated hydrocarbon such as methylene chloride; an ether such as tetrahydrofuran and ethyleneglycol dimethyl ether; a non-protic polar solvent such as N,N-dimethylfolmamide; and others.

An used amount of unsaturated carboxylic acid represented by the formula (3) is, for example, about 0.5 to 50 moles based on one mole of a vinylether compound represented by the formula (4), and preferably about 0.9 to 10 moles. An used amount of acid catalyst is, for example, about 0.0001 to 1 mole based on one mole of a vinylether compound represented by the formula (4), and preferably about 0.001 to 0.3 moles.

In order to prevent a polymerization of a vinylether represented by the formula (4) or a reaction product, adding a little amount of polymerization-inhibiter such as 4-methoxyphenol into the mixture is preferable. An added amount of polymerization-inhibiter is, for example, about 0.00001 to 0.05 mole based on one mole of a vinylether compound represented by the formula (4), and preferably about 0.0001 to 0.01 moles.

A reaction temperature is different corresponding to a sort of raw material for the reaction or a sort of used catalyst and usually −10° C. to 100° C., preferably about 0 to 60° C.

After the reaction a reaction product can be separated and purified by a separating method such as a liquid-property adjustment, a extraction, a concentration, a distillation, a crystallization, a re-crystallization, a column chromatography and so on.

In addition, except for an unsaturated carboxylic acid hemiacetal ester represented by the formula (1), a compound in which both of R^{b} and R^{c} of the formula (1) are a hydrogen atom is also useful as a monomer for a photoresist polymeric compound. A repeated unit corresponding to this compound shows acid-eliminating function and hydrophilic function in the polymeric compound. As such a compound, there may be mentioned a compound corresponding to an example of an unsaturated carboxylic acid hemiacetal ester (a compound which is R^{b}=R^{c}=H) and so on.

A compound in which both of R^{b} and R^{c} of the formula (1) are a hydrogen atom [a compound represented by the formula (B)] can be produced, for example, by reacting an unsaturated carboxylic acid represented by the formula (3) with a halo-methyl ether compound represented by the formula (A) under a base as shown by the following reaction formula. 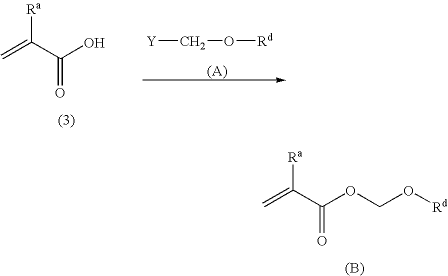
(Wherein each of R^{a} and R^{b} is the same as above. Y is a halogen atom.)

As a halogen atom in Y, there may be mentioned a chlorine atom, a bromine atom, an iodine atom and so on. A reaction is performed in a solvent or without any solvent. As a solvent, the said solvents can be used. As a base, for example, an organic base such as triethylamine and pyridine or an inorganic base such as sodium hydroxide, sodium carbonate and sodium bicarbonate can be used. An used amount of an unsaturated carboxylic acid represented by the formula (3) is, for example, about 0.5 to 10 moles based on one mole of a halo-methyl ether compound, preferably about 0.8 to 2 moles. An used amount of base is, for example, about 1 to 5 moles based on one mole of an unsaturated carboxylic acid represented by the formula (3) and large excess amount may be used. In order to prevent a polymerization of a halo-methyl ether compound or a reaction product, a little of polymerization inhibiter such as 4-methoxy phenol may be added into the mixture. A reaction temperature is usually −10° C. to 100° C., and preferably about 0 to 60° C. After the reaction, a reaction product can be purified by a separating method such as a liquid-property adjustment, a extraction, a concentration, a distillation, a crystallization, a re-crystallization, a column chromatography and so on.

A halo-methyl ether compound represented by the above formula (A) can be produced, for example, by reacting formaldehyde or a same valuable compound thereof (paraformaldehyde, 1,3,5-trioxane and so on) and a hydrogen halide represented by the formula (D) to a hydroxy compound represented by the formula (C) as shown by the following reaction formula. 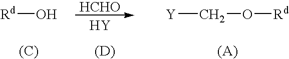
(wherein each of R^{d} and Y is the same above)

As a hydrogen halide represented by the formula (D), there may be mentioned, for example, hydrogen chloride, hydrogen bromide and soon. A reaction is performed in a solvent or without any solvent. As a solvent, the said solvents can be used. An used amount of formaldehyde or a same valuable compound, by changing into formaldehyde, is, for example, about 0.8 to 10 moles based on one mole of a hydroxy compound represented by the formula (C), preferably about 1 to 1.5 moles. An used amount of a hydrogen halide represented by the formula (D) is, for example, about 1 to 5 moles based on one mole of a hydroxy compound represented by the formula (C), and large excess amount may be used. A reaction temperature is usually −10° C. to 100° C., preferably about 0 to 60° C. After the reaction a reaction product can be separated and purified by a separating method such as a liquid-property adjustment, a extraction, a concentration, a distillation, a crystallization, a re-crystallization, a column chromatography and so on.

[Polymeric Compound]

A polymeric compound of the present invention contains a repeated unit (monomer unit) corresponding to the above unsaturated carboxylic acid hemiacetal ester, namely an unit represented by the formula (I). The repeated unit may be one sort or two sorts or more. Such a polymeric compound can be obtained by subjecting the above unsaturated carboxylic acid hemiacetal ester to a polymerization.

Because a repeated unit represented by the formula (I) has a hemi-acetal ester structure, it has acid-eliminating function (alkali soluble function). Namely, because a free carboxyl group is generated by allowing an alcohol portion of ester (hemi-acetal portion) to eliminate by an acid generated from a photo-acid generator during exposure, it becomes to be soluble toward an alkali developer. Further, because three oxygen atoms are contained in a hemi-acetal ester structure, the hydrophilicity is better than a conventional acid-eliminating unit having a simple ester structure (two oxygen atoms is contained) and there is an advantage that a solubility and a wettability are improved toward a resist solvent or an alkali developer. In addition, when a repeated unit represented by the formula (I) has a lactone skeleton, substrate adhesion is excellent. Because a polymeric compound having such a repeated unit having both acid-eliminating function and substrate adhesion function can increase largely substrate adhesion group while keeping a number of acid-eliminating group as compared with a conventional polymeric compound having containing a repeated unit having only acid-eliminating function, it is excellent in the points that high level substrate adhesion is performed and acid-eliminating function, substrate adhesion and other functions are thoroughly improved together by introducing a repeated unit having other functions such as hydrophilicity while keeping a number of acid-eliminating group and a number of substrate adhesion group. Further, when R^{d} in the formula (I) is a group containing a multiple non-aromatic carbon ring (bridged ring), high light transparency and dry-etching resistance are performed.

A polymeric compound of the present invention efficiently has various functions required as a resist in balance, so another repeated unit may be had in additional to a repeated unit represented by the above formula (I). Such another repeated unit can be formed by allowing a polymerizable unsaturated monomer corresponding to the repeated unit to co-polymerize with the said unsaturated carboxylic acid hemiacetal eater. As the above other repeated unit, there may be mentioned, for example, a repeated unit having substrate adhesion and/or hydrophilic function, a repeated unit improving acid-eliminating function, a repeated unit improving etching resistance function, a repeated unit improving transparency and so on. The said hydrophilic function includes a function improving solubility toward a resist solvent or alkali developer. Further, on preparing a polymeric compound of the present invention, a monomer used to perform a co-polymerization smoothly or unify a co-polymer composition can be used as a co-monomer.

A repeated unit having substrate adhesion and/or hydrophilic function can be introduced by using a polymerizable unsaturated monomer having a polar group as a co-monomer. As the said polar group, there may be mentioned, for example, (1) a group such as a group having a lactone ring, a carbonyl group, an acid anhydride group and an imide group, (2) a group such as a hydroxyl group which may have a protecting group, a mercapto group which may have a protecting group, a carboxyl group which may have a protecting group, an amino group which may have a protecting group and a sulfo group which may have a protecting group. In addition, as a polymerizable unsaturated monomer having a polar group, (a) a monomer such as a monomer having a lactone skeleton, a monomer having a cyclic ketone skeleton, a monomer having an acid anhydride group and a monomer having an imide group and (b) a monomer such as a monomer having a hydroxyl group (includes a compound in which a hydroxyl group is protected), a monomer having a mercapto group (includes a compound in which a mercapto group is protected), a monomer having a carboxyl group (includes a compound in which a carboxyl group is protected), a monomer having an amino group (includes a compound in which an amino group is protected) and a monomer having a sulfo group (includes a compound in which a sulfo group is protected) are exemplified and respectively a known compound in the resist field can be used. These monomers can be used alone or by combining two sorts or more. For example, by combining a monomer included in the said (a) with a monomer included the said (b), properties as a resist can be performed in balance.

A repeated unit improving acid-eliminating function can be introduced into a polymer, for example, by using, as a co-monomer, (c) a (meth)acrylic acid ester derivative in which a hydrocarbon group having a tertiary carbon, a 2-tetrahydrofuranyl group, 2-tetrahydropyranyl group or others bonds to an adjacent position of an oxygen atom constituting an ester, (d) a (meth)acrylic acid ester derivative which has a hydrocarbon group (such as an alicyclic hydrocarbon group, an aliphatic hydrocarbon group and a group bonded by these) to an adjacent position of an oxygen atom constituting an ester and is bonded to the hydrocarbon group by —COOR^{x} group (R^{x} is a tertiary hydrocarbon group, a 2-tetrahydrofuranyl group or a 2-tetrahydropyranyl group) directly or through a combining group. As such a (meth)acrylic acid ester derivative, a known compound in the resist field can be used.

As a typical example of a polymerizable unsaturated monomer except for an unsaturated carboxylic acid hemiacetal ester used to give various functions as a resist to a polymeric compound of the present invention, there may be mentioned a compound represented by the formulae (8a) to (8g). These correspond to the said monomer having a lactone skeleton and monomer having a cyclic ketone skeleton. 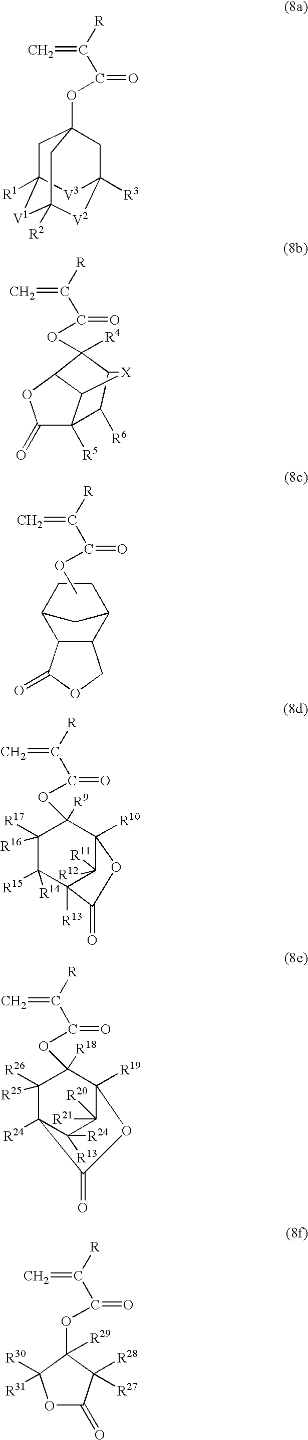 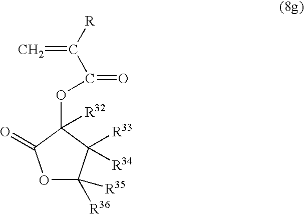
(wherein R is a hydrogen atom or a methyl group. Each of R¹ to R⁶, R⁹ to R³⁶, X and V¹ to V³ is the same as above.)

As a typical example of a compound represented by the formula (8a), there may be mentioned the following compounds and however these aren't limited.
[2-1] 1-(meth)acryloyloxy-4-oxoadamantane (R=H or CH₃, R¹=R²=R³=H, V¹=—CO—, V²=V³=—CH₂—)
[2-2] 1-(meth)acryloyloxy-4-oxatricyclo[4.3.1.1^{3,8}]undecane-5-on (R=H or CH₃, R¹=R=R³=H, V²=—CO—O— (the left side is a carbon atom side bonded by R²), V¹=V³=—CH₂—)
[2-3] 1-(meth)acryloyloxy-4,7-dioxatricyclo[4.4.1.1^{3,9}]dodecane-5,8-dion (R=H or CH₃, R¹=R²=R³=H, V¹=—CO—O— (the left side is a carbon atom side bonded by R¹), V²=CO—O— (the left side is a carbon atom side bonded by R²), V³=—CH₂—)
[2-4] 1-(meth)acryloyloxy-4,8-dioxatricyclo[4.4.1.1^{3,9}]dodecane-5,7-dion (R=H or CH₃, R¹=R²=R³=H, V¹=—CO—O— (the left side is a carbon atom side bonded by R¹), V²=—CO—O— (the left side is a carbon atom side bonded by R²), V³=—CH₂—)
[2-5] 1-(meth)acryloyloxy-5,7-dioxatricyclo[4.4.1.1^{3,9}]dodecane-4,8-dion (R=H or CH₃, R¹=R²=R³=H, V¹=—CO—O— (the left side is a carbon atom side bonded by R¹), V²=—CO—O— (the left side is a carbon atom side bonded by R²), V³=—CH₂—)

As a typical example of a compound represented by the formula (8b), there may be mentioned the following compounds and however these aren't limited.
[2-6] 2-(meth)acryloyloxy-4-oxatricyclo[4.2.1.0^{3,7}]nonane-5-on (=5-(meth)acryloyloxy-2,6-norbornanecarbolactone) (R=H or CH₃, R⁴=R⁵=R⁶=H, X=methylene group)
[2-7] 2-(meth)acryloyloxy-2-methyl-4-oxatricyclo[4.2.1.0^{3,7}]nonane-5-on (R=H or CH₃, R⁴=CH₃, R⁵=R⁶=H, X=methylene group)
[2-8] 2-(meth)acryloyloxy-6-methyl-4-oxatricyclo[4.2.1.0^{3,7}]nonane-5-on (R=H or CH₃, R⁵=CH₃, R⁴=R⁶=H, X=methylene group)
[2-9] 2-(meth)acryloyloxy-9-methyl-4-oxatricyclo[4.2.1.0^{3,7}]nonane-5-on (R=H or CH₃, R⁵=CH₃, R⁴=R⁶=H, X=methylene group)
[2-10] 2-(meth)acryloyloxy-9-carboxy-4-oxatricyclo[4.2.1.0^{3,7}]nonane-5-on (R=H or CH₃, R⁴=R⁵=H, R⁶=COOH, X=methylene group)
[2-11] 2-(meth)acryloyloxy-9-methoxycarbony-4-oxatricyclo[4.2.1.0^{3,7}]nonane-5-on (R=H or CH₃, R⁴=R⁵=H, R⁶=methoxycarbonyl group, X=methylene group)
[2-12] 2-(meth)acryloyloxy-9-ethoxycarbony-4-oxatricyclo[4.2.1.0^{3,7}]nonane-5-on (R=H or CH₃, R⁴=R⁵=H, R⁶=ethoxycarbonyl group, X=methylene group)
[2-13] 2-(meth)acryloyloxy-9-t-butoxycarbony-4-oxatricyclo[4.2.1.0^{3,7}]nonane-5-on (R=H or CH₃, R⁴=R⁵=H, R⁶=t-butoxycarbonyl group, X=methylene group)

As a typical example of a compound represented by the formula (8c), there may be mentioned the following compounds and however these aren't limited.
[2-14] 8-(meth)acryloyloxy-4-oxatricyclo[5.2.1.0^{2,6}]decane-5-on (R=H or CH₃)
[2-15] 9-(meth)acryloyloxy-4-oxatricyclo[5.2.1.0^{2,6}]decane-5-on (R=H or CH₃)

As a typical example of a compound represented by the formula (8d), there may be mentioned the following compounds and however these aren't limited.
[2-16] 4-(meth)acryloyloxy-6-oxabicyclo[3.2.1]octane-7-on (R=H or CH₃, R⁹=R¹⁰=R¹¹=R¹²=R¹³=R¹⁴=R¹⁵=R¹⁶=R¹⁷=H)
[2-17] 4-(meth)acryloyloxy-4-methyl-6-oxabicyclo[3.2.1)octane-7-on (R=H or CH₃, R¹⁰=R¹¹=R¹²=R¹³=R¹⁴=R¹⁵=R¹⁶=R¹⁷=H, R⁹=CH₃)
[2-18] 4-(meth)acryloyloxy-5-methyl-6-oxabicyclo[3.2.1]octane-7-on (R=H or CH₃, R⁹=R¹¹=R¹²=R¹³=R¹⁴=R¹⁵=R¹⁶=R¹⁷=H, R¹=CH₃)
[2-19] 4-(meth)acryloyloxy-4,5-dimethyl-6-oxabicyclo[3.2.1]octane-7-on (R=H or CH₃, R¹¹=R¹²=R¹³=R¹⁴=R¹⁵=R¹⁶=R¹⁷H, R⁹=R¹⁰=CH₃)

As a typical example of a compound represented by the formula (8e), there may be mentioned the following compounds and however these aren't limited.
[2-20] 6-(meth)acryloyloxy-2-oxabicyclo[2.2.2]octane-3-on (R H or CH₃, R¹⁸=R¹⁹=R²⁰=R²¹=R²²=R²³=R²⁴=R²⁵=R²⁶=H)
[2-21] 6-(meth)acryloyloxy-6-methyl-2-oxabicyclo[2.2.2]octane-3-on (R=H or CH₃, R¹⁸=R²⁰=R²¹=R²²=R²³=R²⁴=R²⁵=R²⁶=H, R¹⁹=CH₃)
[2-22] 6-(meth)acryloyloxy-1-methyl-2-oxabicyclo[2.2.2]octane-3-on (R=H or CH₃, R¹⁹=R²⁰=R²¹=R²²=R²³=R²⁴=R²⁵=R²⁶H, R¹⁸=CH₃)
[2-23] 6-(meth)acryloyloxy-1,6-dimethyl-2-oxabicyclo[2.2.2]octane-3-on (R=H or CH₃, R²⁰=R²¹=R²²=R²³=R²⁴=R²⁵=R²⁶H, R¹⁸=R¹⁹=CH₃)

As a typical example of a compound represented by the formula (8f), there may be mentioned the following compounds and however these aren't limited.
[2-24] β-(meth)acryloyloxy-γ-butyrolactone (R=H or CH₃, R²⁷=R²⁸=R²⁹=R³⁰=R³¹=H)
[2-25] β-(meth)acryloyloxy-α,α-dimethyl-γ-butyrolactone (R=H or CH₃, R²⁷=R²⁸=CH₃, R²⁹=R³⁰=R³¹=H)
[2-26] β-(meth)acryloyloxy-γ,γ-dimethyl-γ-butyrolactone (R=H or CH₃, R³⁰=R³¹=CH₃, R²⁷=R²⁸=R²⁹H)
[2-27] β-(meth)acryloyloxy-α,α,β-trimethyl-γ-butyrolactone (R=H or CH₃, R²⁷=R²⁸=R²⁹=CH₃, R³⁰=R³¹=H)
[2-28] β-(meth)acryloyloxy-β,γ,γ-trimethyl-γ-butyrolactone (R=H or CH₃, R²⁹=R³⁰=R³¹=CH₃, R²⁷=R²⁸=H)
[2-29] β-(meth)acryloyloxy-α,α,β,γ,γ-pentamethyl-γ-butyrolactone (R=H or CH₃, R²⁷=R²⁸=R²⁹=R³⁰=R³¹=CH₃)

As a typical example of a compound represented by the formula (8g), there may be mentioned the following compounds and however these aren't limited.
[2-30] α-(meth)acryloyloxy-γ-butyrolactone (R=H or CH₃, R³²=R³³=R³⁴=R³⁵=R³⁶=H)
[2-31] α-(meth)acryloyloxy-α-methyl-γ-butyrolactone (R=H or CH₃, R³²=CH₃, R³³=R³⁴=R³⁵=R³⁶=H)
[2-32] α-(meth)acryloyloxy-β,β-dimethyl-γ-butyrolactone (R=H or CH₃, R³³=R³⁴=CH₃, R³²=R³⁵=R³⁶=H)
[2-33] α-(meth)acryloyloxy-α,β,β-trimethyl-γ-butyrolactone (R=H or CH₃, R³²=R³³=R³⁴=CH₃, R³⁵=R³⁶=H)
[2-34] α-(meth)acryloyloxy-γ,γ-dimethyl-γ-butyrolactone (R=H or CH₃, R³⁵=R³⁶=CH₃, R³²=R³³=R³⁴=H)
[2-35] α-(meth)acryloyloxy-α,γ,γ-trimethyl-γ-butyrolactone (R=H or CH₃, R³²=R³⁵=R³⁶=CH₃, R³³=R³⁴=H)
[2-36] α-(meth)acryloyloxy-β,β,γ,γ-tetramethyl-γ-butyrolactone (R=H or CH₃, R³³=R³⁴=R³⁵=R³⁶=CH₃, R³²=H)
[2-37] α-(meth)acryloyloxy-α,β,β,γ,γ-pentamethyl-γ-butyrolactone (R=H or CH₃, R³²=R³³=R³⁴=R³⁵=R³⁶=CH₃)

As another example of polymerizable unsaturated monomer except for an unsaturated carboxylic acid hemiacetal ester used to give various functions as a resist to a polymeric compound of the present invention, there may be mentioned maleic acid anhydride and maleimide. These correspond to the said monomer having an acid anhydride and monomer having an imide group.

As another typical example of polymerizable unsaturated monomer except for an unsaturated carboxylic acid hemiacetal ester used to give various functions as a resist to a polymeric compound of the present invention, there may be mentioned a compound represented by the formula (9). These compounds corresponds to the said monomer having a hydroxyl group, monomer having a mercapto group, monomer having a carboxyl group, monomer having an amino group, monomer having a sulfonic acid group and monomer having a cyclic ketone skeleton. 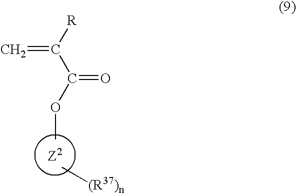
(wherein a ring Z² is an alicyclic hydrocarbon ring of carbon number 6 to 20. R is a hydrogen atom or a methyl group. R³⁷ is a substituent bonding to a ring Z² and is identical to or different from an oxo group, an alkyl group, a hydroxyl group which may be protected by a protecting group, a hydroxyalkyl group which may be protected by a protecting group, a carboxyl group which may be protected by a protecting group, an amino group which may be protected by a protecting group or a sulfonic acid group which may be protected by a protecting group. Provided that at least one of n R³⁷ is an oxo group, a hydroxyl group which may be protected by a protecting group, a hydroxyalkyl group which may be protected by a protecting group, a carboxyl group which may be protected by a protecting group, an amino group which may be protected by a protecting group or a sulfonic acid group which may be protected by a protecting group. n denotes an integer of 1 to 3.

An alicyclic hydrocarbon ring of carbon number 6 to 20 in a ring Z² may be a single ring or a multiple ring such as a condensed ring and a bridged ring. As a typical alicyclic hydrocarbon ring, there may be mentioned, for example, a cyclohexane ring, a cyclooctane ring, a cyclodecane ring, an adamantane ring, a norbornane ring, a norbornene ring, a bornane ring, an isobornane ring, a perhydroindene ring, a decalin ring, a perhydrofuluorene ring (a tricyclo[7.4.0.0^{3,8}]tridecane ring), a perhydroanthracene ring, a tricyclo[5.2.1.0^{2,6}]decane ring, a tricyclo[4.2.2.1^{2,5}]undecane ring, a tetracyclo[4.4.0.1^{2,5}.1^{7,10}]dodecane ring and so on. The alicyclic hydrocarbon ring may have a substituent such as an alkyl group (e.g. an alkyl group of C₁ to C₄ and others) such as methyl group, a haloalkyl group such as a trifluoromethyl group, a halogen atom such as a fluorine atom and a chlorine atom, a hydroxyl group which may be protected by a protecting group, a hydroxyalkyl group which may be protected by a protecting group, a mercapto group which may be protected by a protecting group, an oxo group, a carboxyl group which may be protected by a protecting group, an amino group which may be protected by a protecting group, a sulfonic acid group which may be protected by a protecting group.

In the formula (9), as an alkyl group, there may be mentioned a linear or branched chain alkyl group of carbon number about 1 to 20 such as a methyl, an ethyl, a propyl, an isopropyl, a butyl, an isobutyl, a s-butyl, a t-butyl, a hexyl, an octyl, a decyl and a dodecyl group. As an amino group which may be protected by a protecting group, there may be mentioned an amino group, a substituted amino group (e.g. an alkylamino group of C₁ to C₄ such as a methylamino, an ethylamino, a propylamino group and others) and so on. As a sulfonic acid group which may be protected by a protecting group, there may be mentioned a —SO₃R^{z} group and so on. The said R^{z} is a hydrogen atom and an alkyl group, and as an alkyl group, there may be mentioned a linear or branched chain alkyl group of carbon number about 1 to 6 such as a methyl, an ethyl, a propyl, an isopropyl, a butyl, an isobutyl, a s-butyl, a t-butyl and a hexyl group. In R³⁷, each of a hydroxyl group which may be protected by a protecting group, a hydroxyalkyl group which may be protected by a protecting group, a mercapto group which may be protected by a protecting group and a carboxyl group which may be protected by a protecting group is the same as above.

As a typical example of a compound represented by the formula (9), there may be mentioned the following compounds and however these isn't limited.
[3-1] 1-hydroxy-3-(meth)acryloyloxyadamantane (R=H or CH₃, R³⁷=OH, n=1, Z²=adamantane ring)
[3-2] 1,3-dihydroxy-5-(meth)acryloyloxyadamantane (R=H or CH₃, R³⁷=OH, n=2, Z²=adamantane ring)
[3-3] 1-carboxy-3-(meth)acryloyloxyadamantane (R=H or CH₃, R³⁷=COOH, n=1, Z²=adamantane ring)
[3-4] 1,3-dicarboxy-5-(meth)acryloyloxyadamantane (R=H or CH₃, R³⁷=COOH, n=2, Z²=adamantane ring)
[3-5] 1-carboxy-3-hydroxy-5-(meth)acryloyloxyadamantane (R=H or CH₃, R³⁷=OH, COOH, n=2, Z²=adamantane ring)
[3-6] 1-t-butoxycarbonyl-3-(meth)acryloyloxyadamantane (R=H or CH₃, R³⁷=t-butoxycarbonyl group, n=1, Z²=adamantane ring)
[3-7] 1,3-bis(t-butoxycarbonyl)-5-(meth)acryloyloxyadamantane (R=H or CH₃, R³⁷=t-butoxycarbonyl group, n=2, Z²=adamantane ring)
[3-8] 1-t-butoxycarbonyl-3-hydroxy-5-(meth)acryloyloxyadamantane (R=H or CH₃, R³⁷=OH, t-butoxycarbonyl group, n=2, Z²=adamantane ring)
[3-9] 1-(2-tetrahydropyranyloxycarbonyl)-3-(meth)acryloyloxy adamantane (R=H or CH₃, R³⁷=2-tetrahydropyranyloxycarbonyl group, n=1, Z²=adamantane ring)
[3-10] 1,3-bis(2-tetrahydropyranyloxycarbonyl)-5-(meth)acryloyloxyadamantane (R=H or CH₃, R³⁷=2-tetrahydropyranyloxycarbonyl group, n=2, Z²=adamantane ring)
[3-11] 1-hydroxy-3-(2-tetrahydropyranyloxycarbonyl)-5-(meth) acryloyloxyadamantane (R=H or CH₃, R³⁷=2-tetrahydropyranyloxycarbonyl group, n=2, Z²=adamantane ring)

In addition, as the said monomer having a carboxyl group, for example, acrylic acid, methacrylic acid and others can be used.

As a typical example of a polymerizable unsaturated monomer used to improve acid-eliminating function of a polymeric compound, there may be mentioned, for example, 1-[1-(meth)acryloyloxy-1-methylethyl]adamantane, 2-(meth)acryloyloxy-2-methyladamantane, 2-(meth)acryloyloxy-2-ethyladamantane, 2-[1-(meth)acryloyloxy-1-methylethyl]norbornane, 2-(meth)acryloyloxy-2-methylnorbornane, 1-[1-(meth)acryloyloxy-1-methylethyl]cyclohexane, 1-(meth)acryloyloxy-1-methylcyclohexane, 3-[1-(meth)acryloyloxy-1-methylethyl]tetracyclo[4.4.0.1^{2,5}.1^{7,10}]dodecane, (meth)acrylic acid t-butyl, 1-(t-butoxycarbonyl)-3-(meth)acryloyloxyadamantane, 2-(t-butoxycarbonyl)-5 or 6-(meth)acryloyloxynorbornane, a family group thereof (e.g. a compound bonded by an alkyl group such as an ethyl group or a halo alkyl group such as a trifluoromethyl group exchanging a methyl group) and others.

Further, a corresponding vinyl ether compound in which a (meth)acryloyloxy group in each monomer represented by the above formulae (8a) to (8g) and (9) is substituted by a vinyl group (a vinyl group having a substituent such as a 1-methylvinyl group and a crotyl group is included) can be used as a monomer component of a polymeric compound of the present invention, too.

In a polymeric compound of the present invention, a ratio of a repeated unit represented by the formula (I) isn't particularly limited and however is usually 1 to 100 mole % based on all monomer units constituting a polymer, preferably 10 to 90 mole %, and more preferably about 30 to 80 mole %. A ratio of repeated unit [except for a repeated unit represented by the formula (I)] corresponding to at least one monomer selected from a monomer having a lactone skeleton, a monomer having a cyclic ketone skeleton, a monomer having an acid anhydride and a monomer having an imide group is 0 to 95 mole %, preferably 0 to 60 mole %, and more preferably about 10 to 40 mole %. A ratio of repeated unit corresponding to at least one monomer selected from a monomer having a hydroxyl group, a monomer having a mercapto group and a monomer having a carboxyl group is 0 to 95 mole %, preferably 5 to 90 mole %, and more preferably about 10 to 50 mole %.

In order to obtain a polymeric compound, a polymerization of a monomer mixture can be performed by a common method used to produce an acryl polymer and so on such as a solution polymerization, a bulk polymerization, a suspension polymerization, a bulk-suspension polymerization and a emulsion polymerization and, particularly, a solution polymerization is preferable. Further, in a solution polymerization, a dropwise polymerization is preferable. In detail, a dropwise polymerization may be performed by a method, for example, such as (i) a method in which a monomer solution dissolving in an organic solvent and a polymerization initiator dissolving in an organic solvent are respectively prepared in advance and each of the said monomer solution and polymerization initiator solution are dropped into an organic solvent kept at constant temperature, (ii) a method in which a mixed solution dissolving a monomer and a polymerization initiator in an organic solvent is dropped into an organic solvent kept at constant temperature, (iii) a method in which a monomer solution dissolving in an organic solvent and a polymerization initiator dissolving in an organic solvent are respectively prepared in advance and the said polymerization initiator solution is dropped into the said monomer solution kept at constant temperature and so on.

A known solvent can be used as a polymerization solvent and there may be mentioned, for example, an ether (e.g., a chained ether such as diethylether and a glycolether such as propyleneglycol monomethylether, a cyclic ether such as tetrahydrofuran and dioxane and others), an ester (e.g., methyl acetate, ethyl acetate, butyl acetate, ethyl lactate, a glycolether ester such as propyleneglycol monomethylether acetate and others), a ketone (e.g., acetone, methylethyl ketone, methyl-isobutyl ketone, cyclohexanone and others), an amide (e.g., N,N-dimethylacetoamide, N,N-dimethylformamide and others), a sulfoxide (e.g., dimethylsulfoxide and others), an alcohol (e.g., methanol, ethanol, propanol and others), a hydrocarbon (e.g., an aromatic hydrocarbon such as benzene, toluene and xylene, an aliphatic hydrocarbon such as hexane, an alicyclic hydrocarbon such as cyclohexane and others), a mixed solvent of these and so on. Further, a known polymerization initiator can be used as a polymerization initiator. A polymerization temperature can be properly selected, for example, in a range of about 30 to 150° C.

A polymer obtained by a polymerization can be purified by a precipitation or a re-precipitation. A precipitation or re-precipitation solvent may be either an organic solvent or water and, in addition, may be a mixed solvent. As an organic solvent used as a precipitation or re-precipitation solvent, there may be mentioned, for example, a hydrocarbon (an aliphatic hydrocarbon such as pentane, hexane, heptane and octane; an alicyclic hydrocarbon such as cyclohexane and methylcyclohexane; an aromatic hydrocarbon such as benzene, toluene and xylene), a halogenated hydrocarbon (a halogenated aliphatic hydrocarbon such as methylene chloride, chloroform and carbon tetrachloride; a halogenated aromatic hydrocarbon such as chlorobenzene and dichlorobenzene; and others), a nitro compound (nitromethane, nitroethane and others), a nitrile (acetonitrile, benzonitrile and others), an ether (a chained ether such as diethylether, di-isopropylether and dimethoxyethane; a cyclic ether such as tetrahydrofuran and dioxane), a ketone (acetone, methylethylketone, di-isobutylketone and others), an ester (ethylacetate, butylacetate and others), a carbonate (dimethylcarbonate, diethylcarbonate, ethylenecarbonate, propylenecarbonate and others), an alcohol (methanol, ethanol, propanol, isopropylalcohol, butanol and others), a carboxylic acid (acetic acid and others), a mixed solvent containing these solvents and so on.

Particularly, as an organic solvent used as the said precipitation solvent and re-precipitation solvent, at least a solvent containing a hydrocarbon (particularly an aliphatic hydrocarbon such as hexane) is preferable. In such a solvent containing at least a hydrocarbon, a ratio of a hydrocarbon (particularly an aliphatic hydrocarbon such as hexane) and another solvent is, for example, the former/the latter (volume ratio; 25° C.)=10/90 to 99/1, preferably the former/the latter (volume ratio; 25° C.)=30/70 to 98/2, more preferably the former/the latter (volume ratio; 25° C.)=about 50/50 to 97/3.

A photoresist resin composition of the present invention contains the said polymeric compound of the present invention and a photo-acid generator.

As a photo-acid generator, a conventional compound, namely known, effectively providing acid by the action of exposure, for example, a diazonium salt, an iodonium salt (for example, diphenyl iodo hexafluorophosphate and so on), a sulfonium salt (for example, triphenyl sulfonium hexafluoroantimonate, triphenyl sulfonium hexafluorophosphate, triphenyl sulfonium methane sulfonate and so on), a sulfonic acid ester [for example, 1-phenyl-1-(4-methylphenyl)sulfonyloxy-1-benzoylmethane, 1,2,3-trisulfonyloxymethylbenzene, 1,3-dinitro-2-(4-phenylsulfonyloxymethyl)benzene, 1-phenyl-1-(4-methylphenylsulfonyloxymethyl)-1-hydroxy-1-benzoylmethane and so on], an oxathiazol derivative, s-triazine derivative, a disulfone derivative (such as diphenyldisulfone), an imide compound, an oxime sulfonate, diazonaphtoquinone, benzoin tosylate and others can be applied. These photo-acid generators can be used by alone or in combination of 2 or more sorts.

The amount of photo-acid generator can be appropriately selected depending on strength of the acid generated by photo-exposure, a ratio of each monomer unit (repeated unit) of the polymer and others, and for example, from 0.1 to 30 part by weight, preferably from 1 to 25 part by weight, and preferably from about 2 to 20 part by weight, relative to 100 part by weight of the said polymeric compound.

A photoresist resin composition may contain an alkali soluble component such as an alkali soluble resin (for example, a novolac resin, a phenol resin, an imide resin, carboxyl group-containing resin and soon), a coloring agent (for example, dyes), an organic solvent (for example, such as hydrocarbons, halogenated hydrocarbons, alcohols, esters, amides, ketones, ethers, cellosolves, carbitols, glycol ether esters, and mixed solvent of them) and others.

After the photoresist resin composition is coated on a base or substrate and dried, the applied film (resist film) is exposed to light (or, further baked after exposure) to form a latent pattern, and is subsequently developed to form a fine pattern with a high degree of precision.

As a base or substrate, there may be mentioned silicon wafer, metal, plastics, glass, ceramic and so on. The photoresist resin composition can be applied using a conventional application means such as a spin coater, a dip coater, a roller coater. The applied film has a thickness of, for example, from 0.01 to 20 μm, and preferably from about 0.05 to 2 μm.

Light rays with different wavelengths such as ultraviolet rays and X-rays can be used in exposure and g-light, i-light, excimer laser (for example, XeCl, KrF, KrCl, ArF, ArCl, F₂, Kr₂, KrAr, Ar₂ and so on) are usually used for semiconductor resist. An exposure energy is, for example, about 0.1 to about 1000 mJ/cm².

Light irradiation allows the photosensitive acid generator to generate an acid, and the acid allows, for example, the eliminating portion of acid-eliminating group of the said polymeric compound to leave promptly and thereby yields a carboxyl group, for example, that contributes to solubilization. Therefore, development with water or an alkaline developing solution can yield a predetermined pattern with a high degree of precision.

### EXAMPLES

The present invention will be illustrated in more detail with reference to several examples below, which is not intended to limit the scope of the invention.

### Production Example 1

A mixture of 21.3 g (0.118 mol) of 2-vinyloxy-4-oxatricyclo[4.2.1.0^{3,7}]nonane-5-on represented by the following formula (10), 50.8 g (0.59 mol) of methacrylic acid, 120 mg (12 mmol) of phosphoric acid, 15 mg (0.12 mmol) of 4-methoxyphenol and 210 ml of toluene were placed in 4-necked flask and stirred at 20° C. for 6 hours under nitrogen atmosphere. After the reaction, the reaction mixture was washed respectively twice by 200 ml of 10 weight % sodium carbonate aqueous solution and once by 200 ml of 10 weight % salt aqueous solution and then the organic layers were concentrated under reduced pressure. The concentrated residue was purified by silicagel column chromatography and 25.5 g (96 mmol, yield 81%) of 2-(1-methacryloyloxyethoxy)-4-oxatricyclo[4.2.1.0^{3,7}]nonane-5-on represented by the following formula (12) was obtained. This product is a mixture of two sorts of isomers and its ratio is about 1:1. In addition, 2-vinyloxy-4-oxatricyclo[4.2.1.0^{3,7}]nonane-5-on represented by the formula (10) was synthesized from 2-hydroxy-4-oxatricyclo[4.2.1.0^{3,7}]nonane-5-on represented by the formula (11) and vinyl propionate by using a method described in Japanese Unexamined Patent Application Publication No. 2003-73321 and a product purified by distillation was applied.

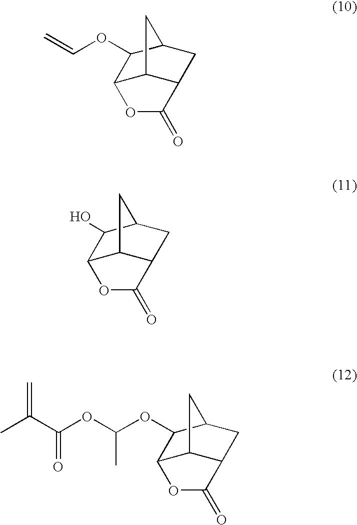

### Spectral data of 2-(1-methacryloyloxyethoxy)-4-oxatricyclo[4.2.1.037]nonane-5-on

¹H-NMR (CDCl₃) δ: 1.40-1.44(m, 3H), 1.56-1.63 (m, 2H), 1.95 (s, 3H), 1.97-2.08(m, 2H), 3.13-3.16(m,1H), 3.59 (m,0.5H), 3.67 (m, 0.5H), 4.49 (d,0.5H), 4.57 (d, 0.5H), 5.62 (m, 1H), 6.05 (m, 1H), 6.14 (m, 1H)

### Production Example 2

In a reaction vessel equipped Dean-Stark apparatus and a thermo meter, 85 g (500 mmol) of 3-hydroxy-1-oxaspiro[4.5]decane-2-on represented by the following formula (13), 31.8 g (300 mmol) of sodium carbonate and 600 ml of toluene were added and heated to 100° C. under nitrogen atmosphere while stirring. 3.36 g (5 mmol) of Ir₂Cl₂(C₈H₁₂)₂ [di-μ-chlorobis(1,5-cyclooctadiene)diiridium(I)] was placed in a reaction vessel and then, while 100 g (1 mol) of vinyl propionate was dropped within two hours, the reaction was performed by heating and refluxing to remove water in azeotropy. After dropping the reaction was continued for more three hours. After the reaction the reaction mixture was cooled by standing, washed by 700 ml of water and concentrated under reduced pressure. By distilling the concentrated residue to purify, 22.5 g (114 mmol, 23%) of colorless and transparent liquid 3-vinyloxy-1-oxaspiro[4.5]decane-2-on represented by the formula (14) was obtained. In addition, 3-hydroxy-1-oxaspiro[4.5]decane-2-on represented by the formula (13) was synthesized from cyclohexanol and methyl acrylate with a method described in the reference [Chem. Commun., 7,613-614(2000)] and purified and it was applied.

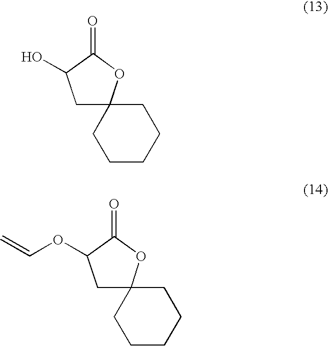

### Spectral Data of 3-vinyloxy-1-oxaspiro[4.5]decane-2-on

¹H-NMR (CDCl₃) δ: 1.35-1.89(m, 10H), 2.04 (dd, 1H), 2.50 (dd, 1H), 4.20(dd, 1H), 4.42(dd,1H), 4.65 (t,1H), 6.48 (q, 1H)

A mixture of 17.1 g (87 mmol) of obtained 3-vinyloxy-1-oxaspiro[4.5]decane-2-on, 37.4 g (435 mmol) of methacrylic acid, 0.85 g (8.7 mmol) of phosphoric acid, 17.1 mg (0.14 mmol) of 4-methoxyphenol and 170 ml of toluene was placed in a reaction vessel and stirred at 50° C. for 4.5 hours under dry air atmosphere. After the reaction, the reaction mixture was washed in order by 170 ml of water, 170 ml of 10 weight % sodium carbonate aqueous solution (twice) and 170 ml of water and the organic layer was concentrated under reduced pressure. The concentrated residue was purified by silicagel column chromatography and 17.3 g (61 mmol, yield 70%) of colorless and transparent liquid 3-(1-methacryloyloxyethoxy)-1-oxaspiro[4.5]decane-2-on [=3-(1-methacryloyloxyethoxy]-2-oxo-1-oxaspiro[4.5]decane] represented by the following formula (15) was obtained. This product was a mixture of an isomer A and an isomer B and its ratio of existence was A:B=about 3:1.

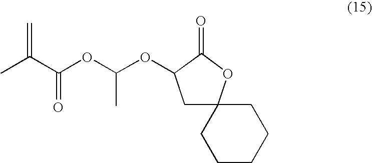

### Spectral data of 3-(1-methacryloyloxyethoxy)-1-oxaspiro[4.5]decane-2-on

Isomer A

¹H-NMR (CDCl₃) δ: 1.34-1.84(m, 13H), 1.92-1.97 (m, 4H), 2.40 (dd, 1H), 4.76(t, 1H), 5.64(m,1H), 6.16 (m,1H), 6.28 (q, 1H)

Isomer B

¹H-NMR (CDCl₃) δ: 1.35-1.86(m, 13H), 1.94-1.99 (m, 4H), 2.53 (dd, 1H), 4.63(t, 1H), 5.64(m,1H), 6.09 (q,1H), 6.21 (m, 1H)

### Production Example 3

A mixture of 43.2 g of adamantane ethanol, 48.1 g of vinyl propionate, 15.3 g of sodium carbonate, 120 ml of toluene and 1.62 g of di-μ-chlorobis(1,5-cyclooctadiene)diiridium (I) was placed in a four-necked flask and stirred for 4 hours under nitrogen atmosphere while heating at 100° C. Precipitates in reaction mixture were filtered and the filtrate was concentrated under reduced pressure. The concentrated mixture was purified by reduced-pressure distillation and 34.8 g of 2-(adamantane-1-yl)ethylvinylether represented by the following formula (16) was obtained.

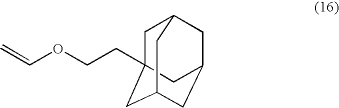

### Spectral data of 2-(adamantane-1-yl)ethylvinylether

¹H-NMR (CDCl₃) δ: 1.46(t, 2H), 1.53 (d, 6H), 1.62-1.72 (m, 6H), 1.95(m, 3H), 3.73(t,2H), 3.96 (m,1H), 4.16 (m, 1H), 6.46 (m, 1H)

### Production Example 4

A mixture of 32.8 g of 2-(adamantane-1-yl)ethylvinylether, 68.4 g of methacrylic acid, 0.16 g of phosphoric acid, 0.164 g of 4-methoxyphenol and 290 ml of toluene was placed in a four-necked flask and stirred at 20° C. for 6 hours under nitrogen atmosphere. After the reaction the reaction mixture was washed respectively by twice of 500 ml of 10 weight % sodium carbonate aqueous solution and once of 500 ml of 10 weight % salt aqueous solution and the organic layer was concentrated under reduced pressure. The concentrated product was purified by silicagel column chromatography and 38.6 g of 1-[2-(adamantane-1-yl)ethoxy]ethyl(meth)acrylate represented by the following formula (17) was obtained.

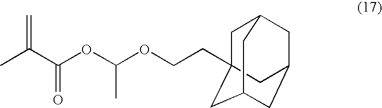

### Spectral data of 1-[2-(adamantane-1-yl)ethoxy]ethyl(meth)acrylate

¹H-NMR (CDCl₃) δ: 1.37-1.41(m, 2H), 1.43 (d, 3H), 1.50 (d, 6H), 1.60-1.71(m, 6H), 1.93(m,3H), 1.96 (m,3H), 3.53 (m, 1H), 3.72(m, 1H), 5.60(m,1H), 5.97 (m,3H), 6.16 (m, 1H)

### Production Example 5

A mixture of 18.0 g (0.1 mol) of bornylvinylether represented by the following formula (18), 43.0 g (0.5 mol) of methacrylic acid, 98 mg (1 mmol) of phosphoric acid, 12 mg (0.1 mmol) of 4-methoxyphenol and 180 ml of toluene was placed in a four-necked flask and stirred at 20° C. for 6 hours under nitrogen atmosphere. After the reaction the reaction mixture was washed respectively by twice of 200 ml of 10 weight % sodium carbonate aqueous solution and once of 200 ml of 10 weight % salt aqueous solution and the organic layer was concentrated under reduced pressure. The concentrated product was purified by silicagel column chromatography and 22.8 g (85 mmol, yield 85%) of 1-(bornyloxy)ethylmethacrylate represented by the following formula (19) was obtained. Further, bornylvinylether was synthesized from (−)-borneol and vinylpropionate by a method described in Japanese Unexamined Patent Application Publication No. 2003-73321 and applied after purifying by a distillation.

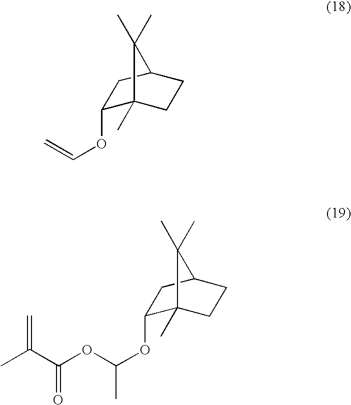

### Spectral data of 1-(bornyloxy)ethylmethacrylate

¹H-NMR (CDCl₃) δ: 0.80-0.84(m, 9H), 0.86 (d, 1H), 1.18-1.27 (m, 2H), 1.42-1.45 (d, 3H), 1.57-1.70 (m, 2H), 1.94(s, 3H), 1.95-1.99 (m, 1H), 2.05-2.20 (m, 1H), 3.79-3.82 (m, 1H), 5.56(m,1H), 5.97-6.00 (m,1H), 6.12 (m, 1H)

### Example 1

Synthesis of a Resin of Following Structure

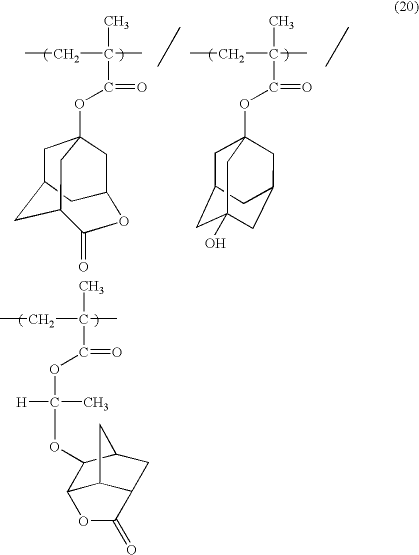

In a separable flask equipped with a stirrer, a thermometer, a dropping funnel and a inlet pipe of nitrogen, 16.5 g of each of propyleneglycol monomethylether acetate (PGMEA) and propyleneglycol monomethylether (PGME) was added and after rising the temperature to 85° C., a mixed solution of 4.93 g of 1-methacryloyloxy-4-oxatricyclo[4.3.1.1^{3,8}]undecane-5-on, 4.66 g of 1-hydroxy-3-methacryloyloxyadamantane, 5.41 g of 2-(1-methacryloloxyethoxy)-4-oxatricyclo[4.2.1.0^{3,7}]nonane-5-on, 0.60 g of dimethyl-2,2′-azobis(2-methylpropionate) (the initiator; made by WAKO JUNYAKU Industry, commercial name “V-601”) and 34.2 g of each of PGMEA and PGME was dropped for 4 hours. After dropping, aging was carried out for 2 hours. The resulting reaction mixture was dropped into a mixed liquid of 733 g of heptane and 81 g of ethyl acetate and the precipitated polymer was recovered with a nuche. The obtained polymer was dried under reduced pressure and 13.8 g of the target product was obtained. In the obtained polymer, the weight average molecular weight (Mw) was 9800 and the molecular-weight distribution (Mw/Mn) was 1.88 (the measured value of GPC, in terms of polystyrene).

### Example 2

Synthesis of a Resin of Following Structure

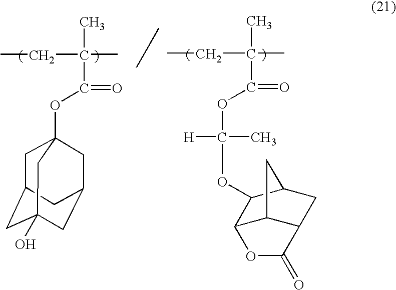

In a separable flask equipped with a stirrer, a thermometer, a dropping funnel and a inlet pipe of nitrogen, 16.5 g of each of propyleneglycol monomethylether acetate (PGMEA) and propyleneglycol monomethylether (PGME) was added and after rising the temperature to 85° C., a mixed solution of 4.13 g of 1-hydroxy-3-methacryloyloxyadamantane, 10.87 g of 2-(1-methacryloloxyethoxy)-4-oxatricyclo[4.2.1.0^{3,7}]nonane-5-on, 0.60 g of dimethyl-2,2′-azobis(2-methylpropionate) (the initiator; made by WAKO JUNYAKU Industry, commercial name “V-601”) and 34.2 g of each of PGMEA and PGME was dropped for 4 hours. After dropping, aging was carried out for 2 hours. The resulting reaction mixture was dropped into a mixed liquid of 733 g of heptane and 81 g of ethyl acetate and the precipitated polymer was recovered with a nuche. The obtained polymer was dried under reduced pressure and 13.5 g of the target product was obtained. In the obtained polymer, the weight average molecular weight (Mw) was 10100 and the molecular-weight distribution (Mw/Mn) was 1.90 (the measured value of GPC, in terms of polystyrene).

### Example 3

Synthesis of a Resin of Following Structure

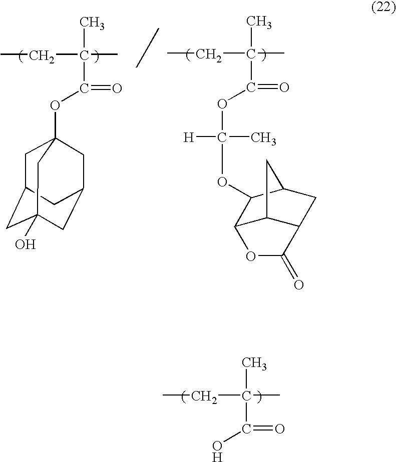

In a separable flask equipped with a stirrer, a thermometer, a dropping funnel and a inlet pipe of nitrogen, 16.5 g of each of propyleneglycol monomethylether acetate (PGMEA) and propyleneglycol monomethylether (PGME) was added and after rising the temperature to 85° C., a mixed solution of 4.44 g of 1-hydroxy-3-methacryloyloxyadamantane, 10.02 g of 2-(1-methacryloloxyethoxy)-4-oxatricyclo[4.2.1.0^{3,7}]nonane-5-on, 0.54 g of methacrylic acid, 0.60 g of dimethyl-2,2′-azobis(2-methylpropionate) (the initiator; made by WAKO JUNYAKU Industry, commercial name “V-601”) and 34.2 g of each of PGMEA and PGME was dropped for 4 hours. After dropping, aging was carried out for 2 hours. The resulting reaction mixture was dropped into a mixed liquid of 733 g of heptane and 81 g of ethyl acetate and the precipitated polymer was recovered with a nuche. The obtained polymer was dried under reduced pressure and 13.5 g of the target product was obtained. In the obtained polymer, the weight average molecular weight (Mw) was 10000 and the molecular-weight distribution (Mw/Mn) was 1.90 (the measured value of GPC, in terms of polystyrene)

### Example 4

Synthesis of a Resin of Following Structure

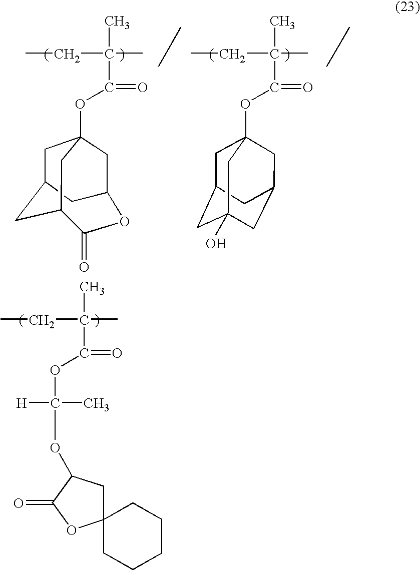

In a separable flask equipped with a stirrer, a thermometer, a dropping funnel and a inlet pipe of nitrogen, 16.5 g of each of propyleneglycol monomethylether acetate (PGMEA) and propyleneglycol monomethylether (PGME) was added and after rising the temperature to 85° C., a mixed solution of 4.88 g of 1-methacryloyloxy-4-oxatricyclo[4.3.1.1^{3,8}]undecane-5-on, 4.61 g of 1-hydroxy-3-methacryloyloxyadamantane, 5.51 g of 3-(1-methacryloyloxyethoxy)-1-oxaspiro[4.5]decane-2-on, 0.60 g of dimethyl-2,2′-azobis(2-methylpropionate) (the initiator; made by WAKO JUNYAKU Industry, commercial name “V-601”) and 34.2 g of each of PGMEA and PGME was dropped for 4 hours. After dropping, aging was carried out for 2 hours. The resulting reaction mixture was dropped into a mixed liquid of 733 g of hexane and 81 g of ethyl acetate and the precipitated polymer was recovered with a nuche. The obtained polymer was drid under reduced pressure and 12.6 g of the target product was obtained. In the obtained polymer, the weight average molecular weight (Mw) was 9900 and the molecular-weight distribution (Mw/Mn) was 1.91 (the measured value of GPC, in terms of polystyrene).

### Example 5

Synthesis of a Resin of Following Structure

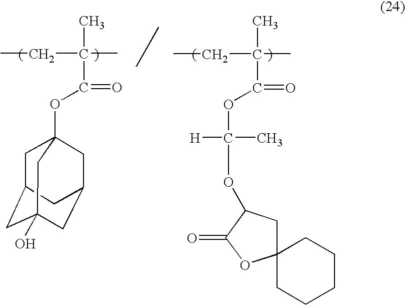

In a separable flask equipped with a stirrer, a thermometer, a dropping funnel and a inlet pipe of nitrogen, 16.5 g of each of propyleneglycol monomethylether acetate (PGMEA) and propyleneglycol monomethylether (PGME) was added and after rising the temperature to 85° C., a mixed solution of 3.96 g of 1-hydroxy-3-methacryloyloxyadamantane, 11.04 g of 3-(1-methacryloyloxyethoxy)-1-oxaspiro[4.5]decane-2-on, 0.60 g of dimethyl-2,2′-azobis(2-methylpropionate) (the initiator; made by WAKO JUNYAKU Industry, commercial name “V-601”) and 34.2 g of each of PGMEA and PGME was dropped for 4 hours. After dropping, aging was carried out for 2 hours. The resulting reaction mixture was dropped into a mixed liquid of 733 g of hexane and 81 g of ethyl acetate and the precipitated polymer was recovered with a nuche. The obtained polymer was dried under reduced pressure and 12.8 g of the target product was obtained. In the obtained polymer, the weight average molecular weight (Mw) was 9800 and the molecular-weight distribution (Mw/Mn) was 1.89 (the measured value of GPC, in terms of polystyrene).

### Example 6

Synthesis of a Resin of Following Structure

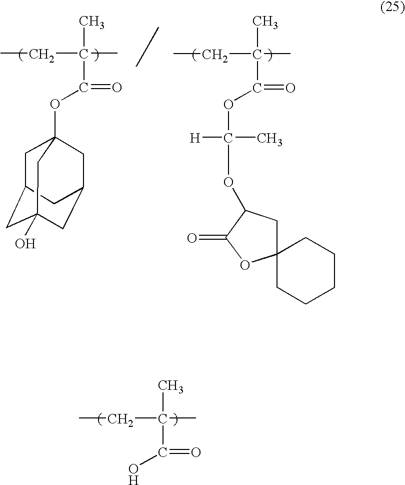

In a separable flask equipped with a stirrer, a thermometer, a dropping funnel and a inlet pipe of nitrogen, 16.5 g of each of propyleneglycol monomethylether acetate (PGMEA) and propyleneglycol monomethylether (PGME) was added and after rising the temperature to 85° C., a mixed solution of 4.27 g of 1-hydroxy-3-methacryloyloxyadamantane, 10.21 g of 3-(1-methacryloyloxyethoxy)-1-oxaspiro[4.5]decane-2-on, 0.52 g of methacrylic acid, 0.60 g of dimethyl-2,2′-azobis(2-methylpropionate) (the initiator; made by WAKO JUNYAKU Industry, commercial name “V-601”) and 34.2 g of each of PGMEA and PGME was dropped for 4 hours. After dropping, aging was carried out for 2 hours. The resulting reaction mixture was dropped into a mixed liquid of 733 g of hexane and 81 g of ethyl acetate and the precipitated polymer was recovered with a nuche. The obtained polymer was dried under reduced pressure and 12.5 g of the target product was obtained. In the obtained polymer, the weight average molecular weight (Mw) was 10100 and the molecular-weight distribution (Mw/Mn) was 1.88 (the measured value of GPC, in terms of polystyrene)

### Example 7

Synthesis of a Resin of Following Structure

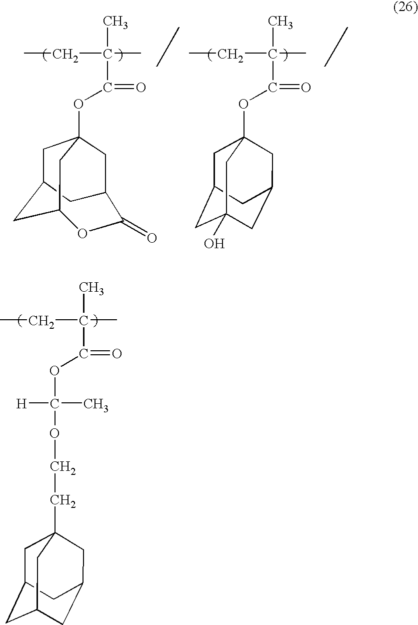

In a separable flask equipped with a stirrer, a thermometer, a dropping funnel and a inlet pipe of nitrogen, 16.5 g of each of propyleneglycol monomethylether acetate (PGMEA) and propyleneglycol monomethylether (PGME) was added and after rising the temperature to 85° C., a mixed solution of 4.77 g of 1-methacryloyloxy-4-oxatricyclo[4.3.1.1^{3,8}]undecane-5-on, 4.50 g of 1-hydroxy-3-methacryloyloxyadamantane, 5.74 g of 1-[2-(adamantane-1-yl)ethoxy]ethylmethacrylate, 0.60 g of dimethyl-2,2′-azobis(2-methylpropionate) (the initiator; made by WAKO JUNYAKU Industry, commercial name “V-601”) and 34.2 g of each of PGMEA and PGME was dropped for 4 hours. After dropping, aging was carried out for 2 hours. The resulting reaction mixture was dropped into a mixed liquid of 733 g of heptane and 81 g of ethyl acetate and the precipitated polymer was recovered with a nuche. The obtained polymer was dried under reduced pressure and 13.5 g of the target product was obtained. In the obtained polymer, the weight average molecular weight (Mw) was 9300 and the molecular-weight distribution (Mw/Mn) was 1.92 (the measured value of GPC, in terms of polystyrene).

### Example 8

Synthesis of a Resin of Following Structure

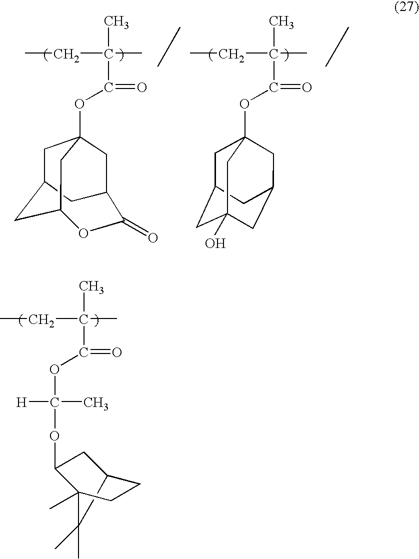

In a separable flask equipped with a stirrer, a thermometer, a dropping funnel and a inlet pipe of nitrogen, 16.5 g of each of propyleneglycol monomethylether acetate (PGMEA) and propyleneglycol monomethylether (PGME) was added and after rising the temperature to 85° C., a mixed solution of 4.93 g of 1-methacryloyloxy-4-oxatricyclo[4.3.1.1^{3,8}]undecane-5-on, 4.66 g of 1-hydroxy-3-methacryloyloxyadamantane, 5.41 g of 1-(bornyloxy)ethylmethacrylate, 0.60 g of dimethyl-2,2′-azobis(2-methylpropionate) (the initiator; made by WAKO JUNYAKU Industry, commercial name “V-601”) and 34.2 g of each of PGMEA and PGME was dropped for 4 hours. After dropping, aging was carried out for 2 hours. The resulting reaction mixture was dropped into a mixed liquid of 733 g of heptane and 81 g of ethyl acetate and the precipitated polymer was recovered with a nuche. The obtained polymer was dried under reduced pressure and 13.2 g of the target product was obtained. In the obtained polymer, the weight average molecular weight (Mw) was 9400 and the molecular-weight distribution (Mw/Mn) was 1.90 (the measured value of GPC, in terms of polystyrene).

### Comparative Example 1

Synthesis of a Resin of Following Structure

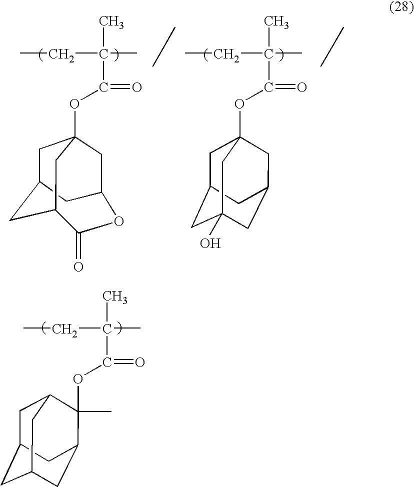

In a separable flask equipped with a stirrer, a thermometer, a dropping funnel and a inlet pipe of nitrogen, 16.5 g of each of propyleneglycol monomethylether acetate (PGMEA) and propyleneglycol monomethylether (PGME) was added and after rising the temperature to 85° C., a mixed solution of 5.16 g of 1-methacryloyloxy-4-oxatricyclo[4.3.1.1^{3,8}]undecane-5-on, 4.87 g of 1-hydroxy-3-methacryloyloxyadamantane, 4.97 g of 2-methacryloyloxy-2-methyladamantane, 0.60 g of dimethyl-2,2′-azobis(2-methylpropionate) (the initiator; made by WAKO JUNYAKU Industry, commercial name “V-601”) and 34.2 g of each of PGMEA and PGME was dropped for 4 hours. After dropping, aging was carried out for 2 hours. The resulting reaction mixture was dropped into a mixed liquid of 733 g of heptane and 81 g of ethyl acetate and the precipitated polymer was recovered with a nuche. The obtained polymer was dried under reduced pressure and 13.5 g of the target product was obtained. In the obtained polymer, the weight average molecular weight (Mw) was 9800 and the molecular-weight distribution (Mw/Mn) was 1.88 (the measured value of GPC, in terms of polystyrene).

Valuation Test

For each of the polymers obtained in the above Examples and Comparative Example, 100 parts by weight of the said polymer and 10 parts by weight of triphenylsulfonium hexafluoroantimonate were mixed with a solvent propyleneglycol monomethyl ether (PGME) to prepare a photoresist resin composition of 17% by weight polymer-concentration. This composition was applied onto a silicon wafer by spin coating method to form a photosensitive layer of 1.0-μm thickness. The photosensitive layer was subjected to prebaking on a hot plate at a temperature of 100° C. for 150 seconds and was exposed to light through a mask using KrF excimer laser having a wavelength of 247 nm at an irradiance of 30 mJ/cm² and then the exposed layer was then subjected to post-exposure baking at a temperature of 100° C. for 60 seconds. Then, it was subjected to development in a 0.3 M aqueous tetramethylammonium hydroxide solution for 60 seconds and was rinsed with pure water. As a result, a pattern with a 0.20-μm line and space was obtained clearly and accurately in any case of using the polymers of Examples but the said pattern was bad accuracy and was lacking in accuracy in case of using the polymer of Comparative Example.

1. An unsaturated carboxylic acid hemiacetal ester represented by the following formula (1); 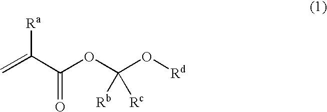
wherein R^{a} is a hydrogen atom, a halogen atom, an alkyl group of carbon number 1 to 6 or a haloalkyl group of carbon number 1 to 6, R^{b} is a hydrocarbon group having a hydrogen atom at a first poison, R^{c} is a hydrogen atom or a hydrocarbon group and R^{d} is an organic group having a cyclic skeleton.

2. An unsaturated carboxylic acid hemiacetal ester according to claim 1, wherein a cyclic skeleton in R^{d} is a lactone skeleton or a non-aromatic polycyclic skeleton.

3. A process of producing an unsaturated carboxylic acid hemiacetal ester, wherein the unsaturated carboxylic acid hemiacetal ester represented by the following formula (5); 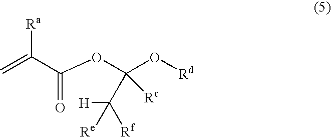
wherein R^{a} is a hydrogen atom, a halogen atom, an alkyl group of carbon number 1 to 6 or a haloalkyl group of carbon number 1 to 6, R^{c} is a hydrogen atom or a hydrocarbon group, R^{d} is an organic group having a cyclic skeleton and each of R^{e} and R^{f} is a hydrogen atom or a hydrocarbon group; is obtained by allowing an unsaturated carboxylic acid represented by the following formula (3); 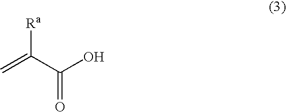
wherein R^{a} is a hydrogen atom, a halogen atom, an alkyl group of carbon number 1 to 6 or a haloalkyl group of carbon number 1 to 6;
to react with a vinyl ether compound represented by the following formula (4); 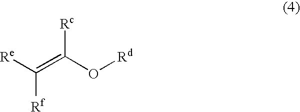
wherein R^{c} is a hydrogen atom or a hydrocarbon group, R^{d} is an organic group having a cyclic skeleton and each of R^{e} and R^{f} is a hydrogen atom or a hydrocarbon group.

4. A polymeric compound having a repeated unit represented by the formula (I); 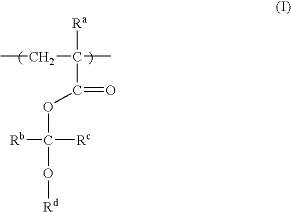
wherein R^{a} is a hydrogen atom, a halogen atom, an alkyl group of carbon number 1 to 6 or a haloalkyl group of carbon number 1 to 6, R^{b} is a hydrocarbon group having a hydrogen atom at a first poison, R^{c} is a hydrogen atom or a hydrocarbon group and R^{d} is an organic group having a cyclic skeleton.

5. A polymeric compound according to claim 4, further having a repeated unit corresponding to at least one monomer selected from a monomer having a lactone skeleton, a monomer having a cyclic ketone skeleton, a monomer having an acid anhydride group and a monomer having an imide group; provided that except for a repeated unit represented by the formula (I).

6. A polymeric compound according to claim 4 or claim 5, further having a repeated unit corresponding to at least one monomer selected from a monomer having a hydroxyl group, a monomer having a mercapto group and a monomer having a carboxyl group.

7. A photoresist resin composition containing at least a polymeric compound described in claim 4 and a photo-acid generator.

8. A process of producing a semi-conductor comprising steps of coating a photoresist resin composition described in claim 7 on a base or substrate to form a resist film and forming a pattern through exposure and development.
